(19) United States
(12) Patent Application Publication
(10) Pub. No.: US 2021/0239963 A1
(43) Pub. Date: Aug. 5, 2021

(54) IMAGING DEVICE, ENDOSCOPE APPARATUS, AND OPERATING METHOD OF IMAGING DEVICE

(71) Applicant: OLYMPUS CORPORATION, Tokyo (JP)

(72) Inventor: Koichiro YOSHINO, Tokyo (JP)

(73) Assignee: OLYMPUS CORPORATION, Tokyo (JP)

(21) Appl. No.: 17/237,481

(22) Filed: Apr. 22, 2021

Related U.S. Application Data
(63) Continuation of application No. PCT/JP2018/041223, filed on Nov. 6, 2018.

(51) Int. Cl. G02B 23/24; G02B 27/10; H04N 5/225

(52) U.S. Cl. CPC G02B 23/2423; G02B 27/1006; H04N 2005/2255; H04N 5/2254; G02B 23/2461

(57) ABSTRACT

An imaging device includes an objective optical system, an optical path splitter splitting an object image into a first and a second optical image different from each other in an in-focus object plane position, an image sensor capturing the first and second optical image to acquire a first and a second image. The processor performs a combining process of selecting an image with a relatively high contrast in predetermined corresponding areas between the first and second image to generate a single combined image. The processor controls the position of a focus lens to be a position determined to form the object image of the target object at a position between a first position corresponding to the image sensor for acquiring the first image and a second position corresponding to the image sensor for acquiring the second image, based on the first and second image before the combining process.

### CROSS REFERENCE TO RELATED APPLICATION

This application is a continuation of International Patent Application No. PCT/JP2018/041223, having an international filing date of Nov. 6, 2018, which designated the United States, the entirety of which is incorporated herein by reference.

### BACKGROUND

An endoscope system is required to have a deepest possible depth of field so as not to impede diagnosis and treatment performed by a user. Despite this requirement, however, the endoscope system recently employs an image sensor having a larger number of pixels, which makes the depth of field shallower. International Publication No. WO 2014/002740 A1 proposes an endoscope system that generates a combined image with an extended depth of field by combining two images that are taken simultaneously at different focal positions. A method of extending a depth of field is hereinafter referred to as an extended depth of field (EDOF) technology.

The endoscope system of International Publication No. WO 2014/002740 A1 further includes a focal point switching mechanism and is configured to be capable of short-distance observation and long-distance observation with the depth of field remaining extended. A design that satisfies a condition that a combined depth of field during the short-distance observation and a combined depth of field during the long-distance observation overlap with each other enables observation of an entire distance range necessary for endoscope observation, without generating a range in which an image is blurred.

### SUMMARY

According to an aspect of the present disclosure, an imaging device includes an objective optical system that includes a focus lens for adjusting an in-focus object plane position and acquires an object image, an optical path splitter that splits the object image into a first optical image and a second optical image different from each other in the in-focus object plane position, an image sensor that captures the first optical image to acquire a first image and the second optical image to acquire a second image, and a processor including hardware. The processor performs a combining process of selecting an image with a relatively high contrast in predetermined corresponding areas between the first image and the second image to generate a single combined image, and an Auto Focus (AF) control of controlling a position of the focus lens to be a position determined to bring a target object into focus. The processor controls the position of the focus lens to be a position determined to form the object image of the target object at a position between a first position corresponding to the image sensor for acquiring the first image and a second position corresponding to the image sensor for acquiring the second image, based on the first image and the second image before the combining process.

According to another aspect of the present disclosure, an endoscope apparatus includes an objective optical system that includes a focus lens for adjusting an in-focus object plane position and acquires an object image, an optical path splitter that splits the object image into a first optical image and a second optical image different from each other in the in-focus object plane position, an image sensor that captures the first optical image to acquire a first image and the second optical image to acquire a second image, and a processor including hardware. The processor performs a combining process of selecting an image with a relatively high contrast in predetermined corresponding areas between the first image and the second image to generate a single combined image, and an Auto Focus (AF) control of controlling a position of the focus lens to be a position determined to bring a target object into focus. The processor controls the position of the focus lens to be a position determined to form the object image of the target object at a position between a first position corresponding to the image sensor for acquiring the first image and a second position corresponding to the image sensor for acquiring the second image, based on the first image and the second image before the combining process.

Still another aspect of the present disclosure relates to an operation method of an imaging device, the imaging device including an objective optical system that includes a focus lens for adjusting an in-focus object plane position and acquires an object image, an optical path splitter that splits the object image into a first optical image and a second optical image different from each other in the in-focus object plane position, and an image sensor that captures the first optical image to acquire a first image and the second optical image to acquire a second image. The method includes a combining process of selecting an image with a relatively high contrast in predetermined corresponding areas between the first image and the second image to generate a single combined image, and an Auto Focus (AF) control of controlling a position of the focus lens to be a position determined to form the object image of a target object at a position between a first position corresponding to the image sensor for acquiring the first image and a second position corresponding to the image sensor for acquiring the second image, based on the first image and the second image before the combining process.

### BRIEF DESCRIPTION OF THE DRAWINGS

FIG. 1 illustrates a configuration example of an imaging device.

FIG. 2 is a diagram for describing a relationship between an image formation position of an object image and a depth of field range.

FIG. 3 illustrates a configuration example of an endoscope apparatus.

FIG. 4 illustrates a configuration example of an imaging section.

FIG. 5 is an explanatory diagram illustrating an effective pixel area of an image sensor.

FIG. 6 illustrates another configuration example of the imaging section.

FIG. 7 illustrates a configuration example of an Auto Focus (AF) control section.

FIG. 8 is a flowchart describing AF control.

FIG. 9 is a flowchart describing a switching process between AF control modes.

FIG. 10 is another flowchart describing the switching process between the AF control modes.

FIG. 11 illustrates another configuration example of the AF control section.

FIGS. 12A to 12C are diagrams for describing relationships between object shapes and desirable combined depth of field ranges.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

Exemplary embodiments are described below. Note that the following exemplary embodiments do not in any way limit the scope of the content defined by the claims laid out herein. Note also that all of the elements described in the present embodiment should not necessarily be taken as essential elements.

1. Overview

International Publication No. WO 2014/002740 A1 or the like discloses an endoscope system that generates a combined image with an extended depth of field by combining two images that are taken simultaneously at different in-focus object plane positions. The in-focus object plane position mentioned herein represents a position of an object, in a case where a system composed of a lens system, an image plane, and an object is in an in-focus state. For example, assuming that the image plane is a plane of an image sensor and that an object image is captured via the lens system using the image sensor, the in-focus object plane position represents a position of an object at which the object is ideally in focus in a captured image. More specifically, in the image captured by the image sensor, the focus is on the object positioned in a depth of field range including the in-focus object plane position. The in-focus object plane position, which is a position at which the object is in focus, may also be referred to as a focal position.

The following description will also mention an image formation position. The image formation position represents a position at which an object image of a given object is formed. The image formation position of the object present at the in-focus object plane position is on a plane of the image sensor. When the position of the object is away from the in-focus object plane position, the image formation position of the object is also away from the plane of the image sensor. In a case where the position of the object is outside the depth of field, an image of the object is captured as a blurred image. The image formation position of the object is a position at which a Point Spread Function (PSF) of the object reaches the peak.

The conventional method as disclosed in International Publication No. WO 2014/002740 A1 or the like assumes a case where focus can be set on an object in a desired range, based on extension of a depth of field by an extended depth of field (EDOF) technology and also based on switching between far point observation and near point observation. However, in a case where the depth of field becomes shallower because of an image sensor having a larger number of pixels, it may be impossible to achieve focus in a certain range by simple switching between the far point observation and the near point observation. For this reason, there is a demand for combining the EDOF technology and Auto Focus (AF) control. However, since an optical system assumed herein is capable of simultaneously capturing a plurality of images that is different in in-focus object plane position, the AF control required herein is not simple application of conventional AF control but execution of more appropriate AF control. In the following description, an image used for the AF control will be discussed first, and thereafter a method of the present embodiment will be described from a first viewpoint of achieving an appropriate depth of field range and a second viewpoint of implementing high-speed AF control.

An imaging device in accordance with the present embodiment simultaneously takes two images that are different in in-focus object plane position, and combines the two images to generate a combined image. That is, the imaging device is capable of acquiring a plurality of images that reflects a state of an object at one given timing. Since images used for the AF control affect a result of the AF control, selection of images to be processed is important for appropriate AF control.

The combined image thus obtained is in a state in which pieces of information about the two images that are different in in-focus object plane position are intricately mixed in accordance with a position on the image. From this combined image, it is extremely difficult to calculate a moving direction or moving amount of a focus lens for the appropriate AF control. Specifically, the appropriate AF control is to control the image formation position of a target object to be a target image formation position.

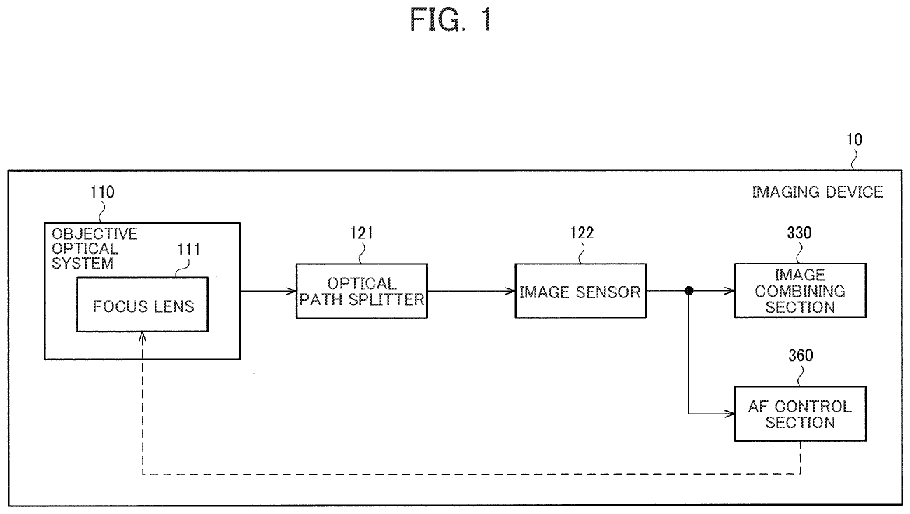

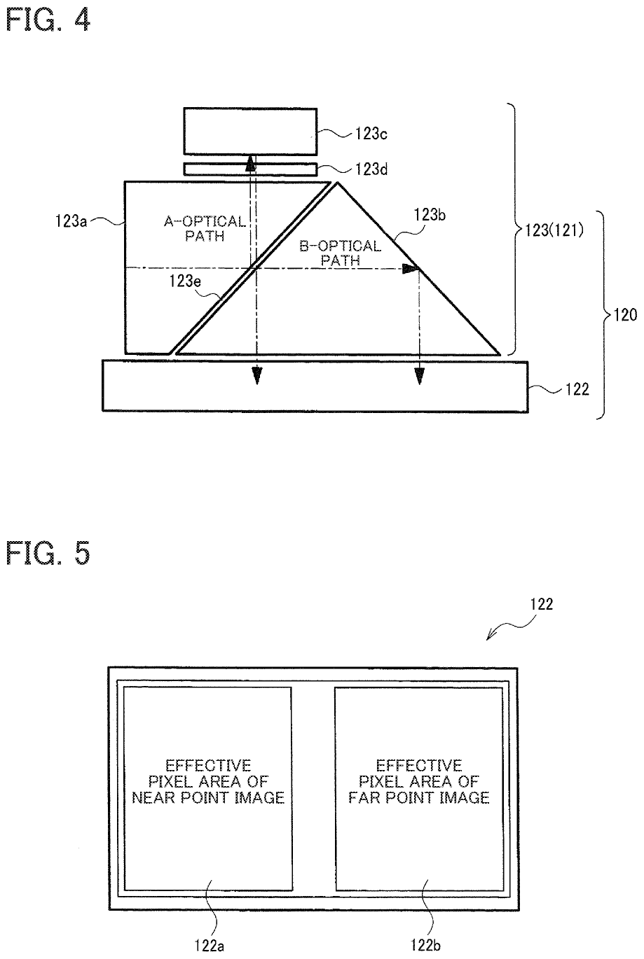

An imaging device 10 in accordance with the present embodiments includes an objective optical system 110, an optical path splitter 121, an image sensor 122, an image combining section 330, and an AF control section 360 as illustrated in FIG. 1. The objective optical system 110 includes a focus lens 111 for adjusting an in-focus object plane position, and acquires an object image. The optical path splitter 121 splits the object image into two optical images that pass through respective two optical paths and that are different in in-focus object plane position. Details of the optical path splitter 121 will be described later with reference to FIGS. 4 to 6. The image sensor 122 captures the first and second optical images that pass through the respective two optical paths to acquire first and second images, respectively. An image that is acquired by capturing the object image that passes through a relatively short optical path and in which an in-focus object plane position is relatively far from the objective optical system 110 is hereinafter referred to as a FAR image. The FAR image may also be referred to as a far point image. In addition, an image that is acquired by capturing the object image that passes through a relatively long optical path and in which an in-focus object plane position is relatively near the objective optical system 110 is hereinafter referred to as a NEAR image. The NEAR image may also be referred to as a near point image. Note that the optical path mentioned herein represents an optical distance in consideration of a refractive index or the like of an object through which light passes. The first image is either of the FAR image or the NEAR image, and the second image is the other one of the FAR image and the NEAR image. As described later with reference to FIGS. 4 to 6, the image sensor 122 may be one element or may include a plurality of elements.

The image combining section 330 performs a combining process of selecting an image with a relatively high contrast in predetermined corresponding areas between the first image and the second image to generate a single combined image. The AF control section 360 controls the position of the focus lens 111 to be a position determined to bring the target object into focus. In this context, “in(to) focus” means that the target object is positioned within the depth of field range.

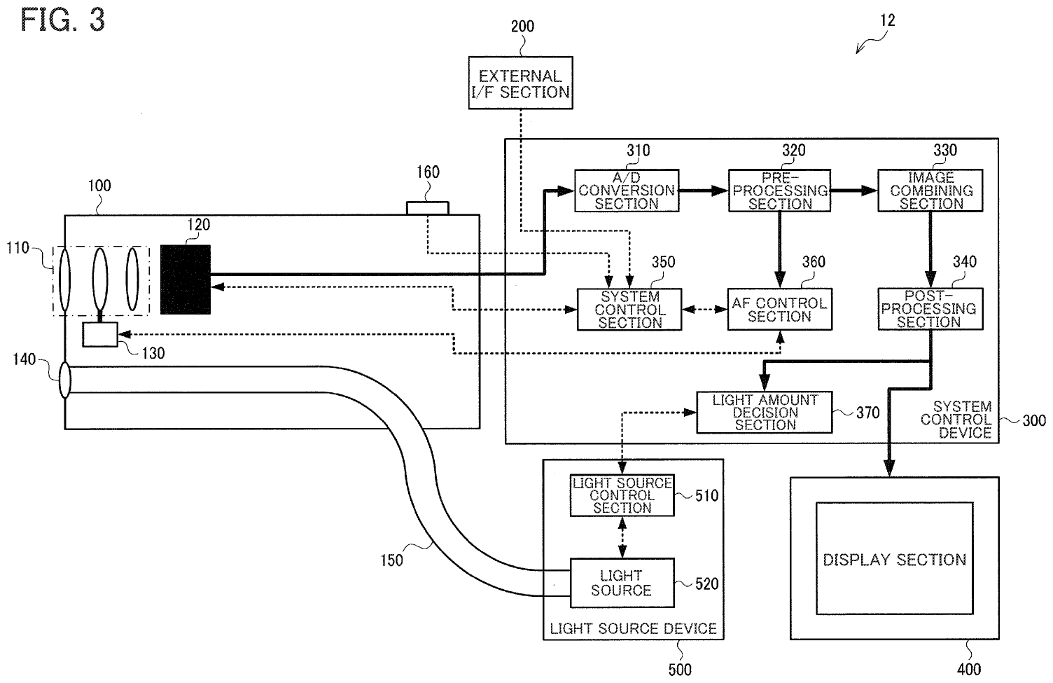

The AF control section 360 performs the AF control, based on at least one of the first image or the second image before the combining process in the image combining section 330. In the first image, the in-focus object plane position is constant at any position on the first image. Similarly, in the second image, the in-focus object plane position is constant at any position on the second image. In generating a combined image from a plurality of simultaneously captured images, the method of the present embodiment enables the AF control using an appropriate image. Note that the first and second images are only required to be images before the combining process, and may be subjected to image processing other than the combining process. For example, the AF control section 360 may perform the AF control using an image subjected to preprocessing by a preprocessing section 320 as described later with reference to FIG. 3. Alternatively, the AF control section 360 may perform the AF control using an image before the preprocessing.

Subsequently, the method of the present embodiment will be described from the first viewpoint. According to the conventional AF control, the lens position of the focus lens 111 is controlled to be a position determined to form the object image of the target object on the image sensor. However, for the imaging device 10 which simultaneously captures the two images that are different in in-focus object plane position and which generates the combined image using these images, the control for setting the image formation position on the image sensor is not always desirable.

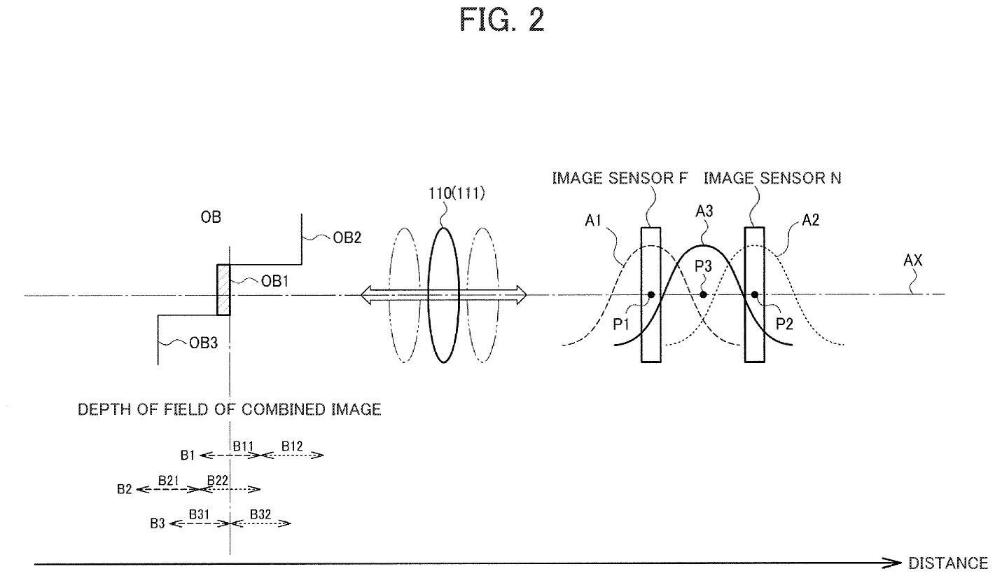

FIG. 2 is a diagram for describing a relationship between the image formation position of the given object and the depth of field of the combined image. Note that FIG. 2 illustrates the focus lens 111 among other elements of the objective optical system 110. The optical path splitter 121 splits the object image into respective two optical images that pass through a relatively short optical path from the objective optical system 110 to the image sensor 122 and a relatively long optical path from the objective optical system 110 to the image sensor 122. FIG. 2 expresses the two optical paths on one optical axis AX. Optical path splitting by the optical path splitter 121 is synonymous with arrangement of two image sensors 122 at different positions on the optical axis AX. The two image sensors 122 are, for example, an image sensor F and an image sensor N illustrated in FIG. 2.

The image sensor F is an image sensor on which the object image that passes through a relatively short optical path is formed, and captures the FAR image in which the in-focus object plane position is far from a given reference position. The image sensor N is an image sensor on which the object image that passes through a relatively long optical path is formed, and captures the NEAR image in which the in-focus object plane position is near the given reference position. The reference position mentioned herein is a position serving as a reference in the objective optical system 110. The reference position may be a position of a fixed lens that is the nearest to the object among other elements of the objective optical system 110, a distal end position of an insertion section 100, or another position. Note that the two image sensors F and N may be implemented by one sheet of the image sensor 122 as described later with reference to FIG. 3.

In FIG. 2, OB represents objects, and OB1 represents the target object among the objects OB. The target object represents an object that is determined as drawing user's attention among the objects. In a case where the imaging device 10 is an endoscope apparatus 12, the target object is a lesion, for example. However, the target object is only required to be an object desired by the user for attentive observation, and is not limited to a lesion. For example, the target object may be bubbles, residues, etc., depending on the purpose of observation. The target object may be designated by the user, or may be automatically set using a known lesion detection method or the like.

During endoscopic visual examination, the user determines a type and malignancy of the lesion, extent of the lesion, and the like by observing not only the lesion as the target object, but also a structure surrounding the lesion. Also for the target object other than the lesion, it is important to observe a peripheral area of the target object. For example, OB2 and OB3 illustrated in FIG. 2 are desirably within a combined depth of field range. Additionally, in a case where a positional relationship between the insertion section 100 and the objects OB changes, it is not desirable for OB2 and OB3 to be outside a combined depth of field immediately.

Now, consider a case of forming the object image of the target object on the image sensor, similarly to the conventional method. In a case of forming the object image of the target object OB1 on the image sensor F, the PSF of the target object OB1 is A1, and the depth of field of the combined image has a range indicated by B1. The depth of field of the combined image, indicated by B1, has a combined range of a depth of field (B11) corresponding to the image sensor F and a depth of field (B12) corresponding to the image sensor N. For convenience of explanation, B11 and B12 are illustrated with an equal width in FIG. 2, but the depth of field normally becomes wider toward the far point side. In a case where the depth of field range is the range indicated by B1, the combined image becomes an unbalanced image, with an object in a direction near the objective optical system 110 with respect to the target object being in focus in a wide range, and with an object in a direction far from the objective optical system 110 with respect to the target object being in focus in a relatively narrow range. That is, the state in which the image formation position is on the image sensor F, as indicated by A1, is not necessarily appropriate for observation including a peripheral object around the target object.

In addition, in a case of forming the object image of the target object on the image sensor N, the PSF of the target object is A2, and the depth of field of the combined image has a range B2 as a combination of B21 and B22. In a case where the depth of field range is the range indicated by B2, the combined image becomes an unbalanced image, with the object in the direction near the objective optical system 110 with respect to the target object being in focus in a narrow range, and with the object in the direction far from the objective optical system 110 with respect to the target object being in focus in a relatively wide range.

Desirably, the present embodiment provides the combined image, with both of the object in the direction near the objective optical system 110 with respect to the target object and the object in the direction far from the objective optical system 110 with respect to the target object being in focus in a balanced manner. Hence, the AF control section 360 controls the position of the focus lens 111 to be a position determined to form the object image of the target image at a position between a first position corresponding to the image sensor 122 that acquires the first image and a second position corresponding to the image sensor 122 that acquires the second image.

The position corresponding to the image sensor 122 mentioned herein is a position determined based on an optical action by the optical path splitter 121, and is different from a physical position at which the image sensor 122 is arranged in the imaging device 10. For example, the first position is a position determined based on the relatively short optical path length of one of the two optical images split by the optical path splitter 121. The second position is a position determined based on the relatively long optical path length of the other of the two optical images split by the optical path splitter 121. In other words, the first position is the image formation position of an image of a given object when the given object has come ideally in focus in the first image. Similarly, the second position is the image formation position of an image of a given object when the given object has come ideally in focus in the second image. In the example illustrated in FIG. 2, the first position corresponds to P1, and the second position corresponds to P2. Instead, the first position may be the position corresponding to the long optical path length, and the second position may be the position corresponding to the short optical path length.

The AF control section 360 in the present embodiment controls the position of the focus lens 111 to be such a position that the PSF of the target object is A3. That is, the AF control section 360 controls the lens position of the focus lens 111 to be such a position that the image formation position of the object image of the target object is P3 between P1 and P2. In this case, the depth of field of the combined image has a range B3 as a combination of B31 and B32. The AF control for forming the object image of the target object at an intermediate position between the image sensor F and the image sensor N enables acquisition of the combined image, with both of the object in the direction near the objective optical system 110 with respect to the target object and the object in the direction far from the objective optical system 110 with respect to the target object being in focus in a balanced manner.

In the example illustrated in FIG. 2, the target object OB1 having a planer structure is observed in a perpendicular direction. Additionally, it is conceivable that the target object OB1 is observed obliquely, or that the target object itself has a depth such as an object having projections and depressions. Also in this case, it is important to ensure a balance of the combined depth of field range, and thus is desirable that the image formation position corresponding to a given portion of the target object should be set between the first and second positions.

Note that FIG. 2 illustrates a case of forming the object image at a center position that is at an equal distance from the image sensor F and the image sensor N. In reality, however, the range of the depth of field changes non-linearly in accordance with the in-focus object plane position. Specifically, the farther the in-focus object plane position is from the objective optical system 110, the wider the depth of field becomes. Thus, a state in which the object image is formed at the center position between the image sensor F and the image sensor N is not always the most balanced in-focus state. For this reason, the image formation position of the object image may be adjusted to a freely-selected position between the image sensor F and the image sensor N. Alternatively, the present embodiment may be capable of adjusting a final image formation position in accordance with user's preference, for example, from the external I/F section 200.

Subsequently, the method of the present embodiment will be described from the second viewpoint. According to the conventional AF control, a well-known technique is to search for the peak of an AF evaluation value calculated from a captured image. For example, in using contrast AF, the AF evaluation value is a contrast value. A peak searching process involves, for example, a process of discriminating an in-focus direction by capturing a plurality of images that is different in in-focus object plane position and comparing AF evaluation values calculated from the respective images. The in-focus direction represents a moving direction of the focus lens 111 determined to increase a focusing degree of the target object. To capture a plurality of images that is different in in-focus object plane position, the conventional method needs to capture the images at a plurality of different timings while changing the position of the focus lens or image sensor.

In the method of the present embodiment, on the other hand, the AF control section 360 operates in accordance with a given AF control mode to control the position of the focus lens 111 to be a position determined to bring the target object into focus. As the AF control mode, the AF control section 360 includes a first AF control mode of performing the AF control using a first AF evaluation value calculated from the first image and a second AF evaluation value calculated from the second image. Specifically, the AF control section 360 is operable in the AF control mode using both of an AF evaluation value of the FAR image captured by the image sensor F at a given timing and an AF evaluation value of the NEAR image captured by the image sensor N at the same timing.

The method of the present embodiment enables acquisition and comparison of a plurality of AF evaluation values based on a result of capturing images at a given one timing. As compared to the conventional method, the method of the present embodiment enables discrimination of the in-focus direction in a shorter time, thereby enabling higher-speed AF control.

In addition, the conventional method determines whether or not the target object is in focus, depending on whether or not the AF evaluation value has reached the peak. Since the determination of whether or not the value has reached the peak cannot be made from an absolute value of the AF evaluation value, comparison with peripheral AF evaluation values is essential. In contrast, the present embodiment enables determination of whether the focusing operation has been completed, based on a relationship between two AF evaluation values. That is, the present embodiment can speed up not only the discrimination of the in-focus direction, but also the determination of whether or not the focusing operation has been completed.

2. System Configuration

While a description will be given below of a case where the imaging device 10 of the present embodiment is the endoscope apparatus 12, the imaging device 10 is not limited to the endoscope apparatus 12. The imaging device 10 is only required to be an apparatus for generating a combined image by capturing a plurality of images that is different in in-focus object plane position and for executing AF control. The imaging device 10 may be, for example, a microscope.

FIG. 3 illustrates a detailed configuration example of the endoscope apparatus 12. The endoscope apparatus 12 includes an insertion section 100, an external interface (I/F) section 200, a system control device 300, a display section 400, and a light source device 500.

The insertion section 100 is a portion to be inserted into the body. The insertion section 100 includes the objective optical system 110, an imaging section 120, an actuator 130, an illumination lens 140, a light guide 150, and an AF start/end button 160.

The light guide 150 guides illumination light emitted from a light source 520 to a distal end of the insertion section 100. The illumination lens 140 emits illumination light guided by the light guide 150 to an object. The objective optical system 110 receives the reflected light from the object and forms an image as an object image. The objective optical system 110 includes the focus lens 111, and is capable of changing the in-focus object plane position in accordance with the position of the focus lens 111. The actuator 130 drives the focus lens 111 based on an instruction from the AF control section 360.

The imaging section 120 includes the optical path splitter 121 and the image sensor 122, and simultaneously acquires the first and second images that are different in in-focus object plane position. The imaging section 120 sequentially acquires a set of the first and second images. The image sensor 122 may be a monochrome sensor or a sensor having a color filter. The color filter may be a well-known Bayer filter, a complementary color filter, or another filter. The complementary color filter includes color filters of cyan, magenta, and yellow separately.

FIG. 4 is a diagram illustrating a configuration example of the imaging section 120. The imaging section 120 is arranged on a rear end side of the insertion section 100 of the objective optical system 110. The imaging section 120 includes a polarizing beam splitter 123 that splits the object image into two optical images that are different in in-focus object plane position, and the image sensor 122 that acquires two images by capturing two optical images. That is, in the imaging section 120 illustrated in FIG. 4, the optical path splitter 121 is the polarizing beam splitter 123.

The polarizing beam splitter 123 includes a first prism 123a, a second prism 123b, a mirror 123c, and a λ/4 plate 123d, as illustrated in FIG. 4. The first prism 123a and the second prism 123b each include a beam split plane having a gradient of 45 degrees with respect to the optical axis. A polarization separation film 123e is arranged on the beam split plane of the first prism 123a. The first prism 123a and the second prism 123b constitute the polarizing beam splitter 123, with their beam split planes being placed in contact with each other across the polarization separation film 123e. The mirror 123c is arranged near an end surface of the first prism 123a, and the λ/4 plate 123d is arranged between the mirror 123c and the first prism 123a. The image sensor 122 is attached to an end surface of the second prism 123b.

The object image from the objective optical system 110 is separated into a P-component and an S-component by the polarization separation film 123e arranged on the beam split plane of the first prism 123a, and separated into two optical images, one being an image on a reflected light side and the other being an image on a transmitted light side. The P-component corresponds to transmitted light, and the S-component corresponds to reflected light.

The optical image of the S-component is reflected by the polarization separation film 123e to the opposite side of the image sensor 122, transmitted along an A-optical path through the λ/4 plate 123d, and thereafter turned back toward the image sensor 122 by the mirror 123c. The turned back optical image is transmitted through the λ/4 plate 123d again, which rotates its polarization direction by 90°. The optical image is transmitted further through the polarization separation film 123e, and thereafter formed on the image sensor 122.

The optical image of the P-component is transmitted through the polarization separation film 123e along a B-optical path, and reflected by a mirror plane of the second prism 123b, the mirror plane being arranged on the opposite side of the beam split plane and turning light vertically toward the image sensor 122, so that the optical image is formed on the image sensor 122. In this case, the A and B optical paths are arranged to have a predetermined optical path difference therebetween, for example, to an extent of several tens of micrometers, thereby allowing two optical images that are different in focal position to be formed on the light receiving plane of the image sensor 122.

As illustrated in FIG. 5, two light receiving areas 122a and 122b are arranged in a whole pixel area in the image sensor 122. The light receiving area may be also referred to as an effective pixel area. To capture the two optical images, the light receiving areas 122a and 122b are positioned to match corresponding image formation planes of the optical images. In the image sensor 122, the in-focus object plane position in the light receiving area 122a is shifted to the near point side relative to the light receiving area 122b. With this arrangement, the two optical images that are different in in-focus object plane position are formed on the light receiving plane of the image sensor 122.

In the example illustrated in FIGS. 4 and 5, the light receiving area 122a of the image sensor 122 corresponds to the image sensor N that captures the NEAR image. In addition, the light receiving area 122b of the image sensor 122 corresponds to the image sensor F that captures the FAR image. That is, in the example illustrated in FIGS. 4 and 5, the image sensor N and the image sensor F are implemented by one sheet of element.

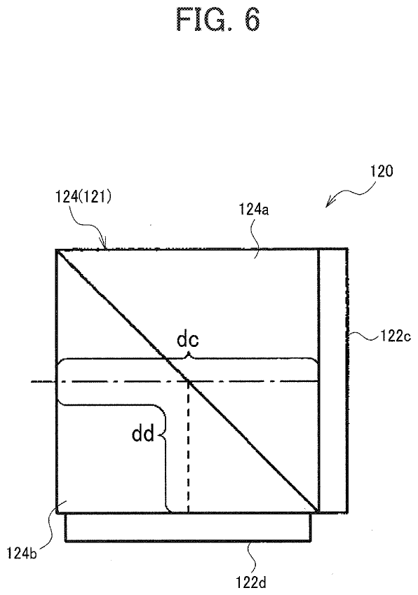

FIG. 6 is a diagram illustrating another configuration example of the imaging section 120. As illustrated in FIG. 6, the imaging section 120 includes a prism 124 and two image sensors 122. Specifically, the two image sensors 122 are image sensors 122c and 122d. In the imaging section 120 illustrated in FIG. 6, the optical path splitter 121 is the prism 124.

This prism 124 is formed, for example, of right-angled triangular prism elements 124a and 124b with their inclined surfaces placed in contact with each other. The image sensor 122c is mounted near, and face to face with, an end surface of the prism element 124a. The other image sensor 122d is mounted near, and face to face with, an end surface of the prism element 124b. Preferably, the image sensors 122c and 122d have uniform characteristics.

When light is incident on the prism 124 via the objective optical system 110, the prism 124 separates the light into reflected light and transmitted light, for example, in an equal amount of light, thereby separating an object image into two optical images, one being an image on a transmitted light side and the other being an image on a reflected light side. The image sensor 122c photoelectrically converts the optical image on the transmitted light side, and the image sensor 122d photoelectrically converts the optical image on the reflected light side.

In the present embodiment, the image sensors 122c and 122d are different in in-focus object plane position. For example, in the prism 124, an optical path length dd on the reflected light side is shorter (smaller) than an optical path length (path length) dc to the image sensor 122c on the transmitted light side. The in-focus object plane position of the image sensor 122c is shifted to the near point side relative to that of the image sensor 122d. It is also possible to change the optical path lengths to the image sensors 122c and 122d by differentiating refractive indexes of the prism elements 124a and 124b. In the example illustrated in FIG. 6, the image sensor 122c corresponds to the image sensor N that captures the NEAR image, and the image sensor 122d corresponds to the image sensor F that captures the FAR image. That is, in the example illustrated in FIG. 6, the image sensor N and the image sensor F are implemented by two sheets of elements.

As illustrated in FIGS. 4 to 6, the specific configuration of the imaging section 120 can be modified in various manners. In addition, the imaging section 120 is only required to be capable of acquiring the first and second images by capturing respective object images that pass through respective two optical paths that are different in in-focus object plane position. Thus, the imaging section 120 is not limited to the configuration exemplified using FIGS. 4 to 6.

The AF start/end button 160 is an operation interface that allows a user to operate the start/end of AF.

The external I/F section 200 is an interface to accept an input from the user to the endoscope apparatus 12. The external I/F section 200 includes, for example, an AF control mode setting button, an AF area setting button, and an image processing parameter adjustment button.

The system control device 300 performs image processing and controls the entire system. The system control device 300 includes an analog to digital (A/D) conversion section 310, the preprocessing section 320, the image combining section 330, a postprocessing section 340, a system control section 350, the AF control section 360, and a light amount decision section 370.

The system control device 300 (a processing section and a processing circuit) in accordance with the present embodiment is composed of the following hardware. The hardware can include at least one of a digital signal processing circuit or an analog signal processing circuit. For example, the hardware can be composed of one or more circuit elements mounted on a circuit board, or one or more circuit elements. The one or more circuit elements are, for example, an integrated circuit (IC) or the like. The one or more circuit elements are, for example, a resistor, a capacitor, or the like.

In addition, the processing circuit, which is the system control device 300, may be implemented by the following processor. The imaging device 10 of the present embodiment includes a memory that stores information, and a processor that operates based on the information stored in the memory. The information is, for example, a program and various kinds of data. The processor includes hardware. Various types of processors such as a central processing unit (CPU), a graphics processing unit (GPU), and a digital signal processor (DSP) may be used as the processor. The memory may be a semiconductor memory such as a static random access memory (SRAM) and a dynamic random access memory (DRAM), or may be a register. The memory may be a magnetic storage device such as a hard disk drive (HDD), or may be an optical storage device such as an optical disk device. For example, the memory stores a computer-readable command, and the processor executes the command to implement a function of each section of the imaging device 10 as processing. Each section of the imaging device 10 is, more specifically, each section of the system control device 300, and includes the A/D conversion section 310, the preprocessing section 320, the image combining section 330, the postprocessing section 340, the system control section 350, the AF control section 360, and the light amount decision section 370. The command may be a command set that is included in a program, or may be a command that instructs the hardware circuit included in the processor to operate.

Each section of the system control device 300 in the present embodiment may be implemented as a module of a program that operates on the processor. For example, the image combining section 330 is implemented as an image combining module, and the AF control section 360 is implemented as an AF control module.

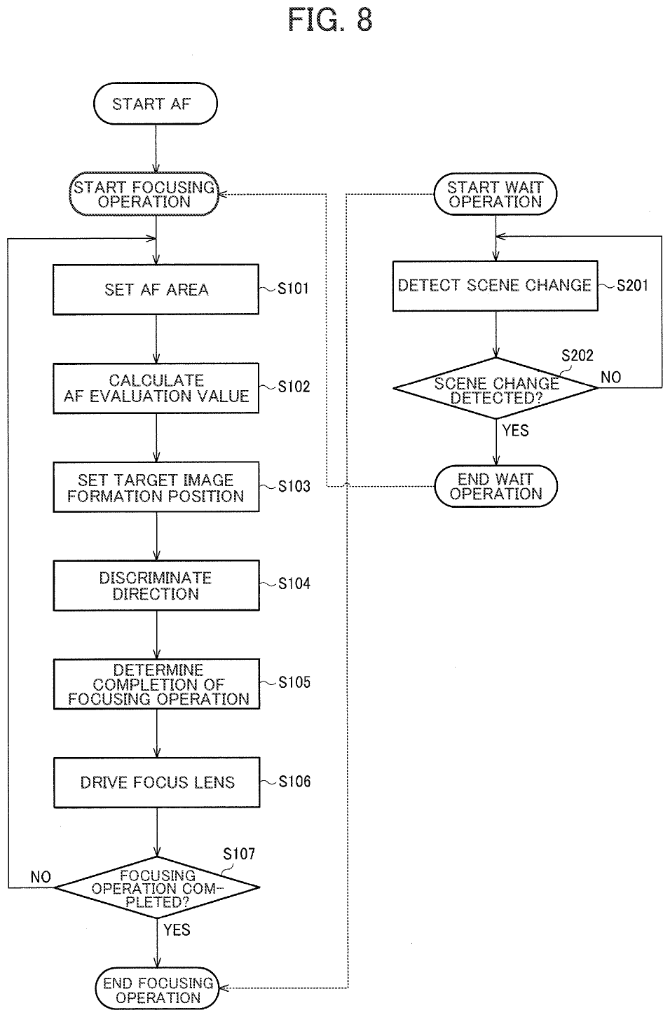

In addition, the program implementing the processing performed by each section of the system control device 300 in the present embodiment can be stored, for example, in an information storage device, which is a computer-readable information storage medium. The information storage device can be implemented by, for example, an optical disk, a memory card, an HDD, or a semiconductor memory. The semiconductor memory is, for example, a read-only memory (ROM). The system control device 300 performs various kinds of processing of the present embodiment, based on the program stored in the information storage device. That is, the information storage device stores the program causing a computer to function as each section of the system control device 300. The computer is a device including an input device, a processing section, a storage section, and an output section. The program causes the computer to execute the processing of each section of the system control device 300. Specifically, the program in accordance with the present embodiment causes the computer to execute each step, which will be described later with reference to FIGS. 8 to 10.

The A/D conversion section 310 converts analog signals, which are sequentially output from the imaging section 120, into digital images and sequentially outputs the digital images to the preprocessing section 320. The preprocessing section 320 performs various types of correction processing on the FAR and NEAR images sequentially output from the A/D conversion section 310, and sequentially outputs the resultant images to the image combining section 330 and the AF control section 360. In a case of separating the object image into two images and thereafter forming the two images on the image sensor, the following geometric differences may occur: the two object images formed on the imaging plane of the image sensor 122 are mismatched between each other in magnification, position, and rotational direction. Additionally, in a case of using the two image sensors 122c and 122d as the image sensor 122, brightness may be mismatched due to, for example, a difference in sensitivity between the sensors. When any of such mismatches is excessive, the combined image will be a double image, will have an unnatural, uneven brightness, or will suffer from other defects. Hence, the present embodiment corrects these geometric differences and brightness difference in the preprocessing section 320.

The image combining section 330 combines corrected two images sequentially output from the preprocessing section 320 to generate a single combined image, and sequentially outputs the combined image to the postprocessing section 340. Specifically, in predetermined corresponding areas of the two images corrected by the preprocessing section 320, the image combining section 330 performs a process of selecting an image with a relatively high contrast to generate the combined image. That is, the image combining section 330 compares respective contrasts in spatially identical pixel areas in the two images, selects a pixel area with a relatively higher contrast, and thereby combines the two images to generate the single combined image. Note that in a case where the contrasts between the identical pixel areas in the two images have a small difference or are substantially equal, the image combining section 330 may perform a process of assigning predetermined weights to the contrasts in the pixel areas and thereafter adding the weighted contrasts to generate a combined image.

The postprocessing section 340 performs various kinds of image processing such as white balance processing, demosaicing processing, noise reduction processing, color conversion processing, gray scale conversion processing, and contour enhancement processing, on the combined image sequentially output from the image combining section 330. Thereafter, the postprocessing section 340 sequentially outputs the combined image to the light amount decision section 370 and the display section 400.

The system control section 350 is connected to the imaging section 120, the AF start/end button 160, the external I/F section 200, and the AF control section 360, and controls each section. Specifically, the system control section 350 inputs/outputs various kinds of control signals. The AF control section 360 performs the AF control using at least one of the corrected two images sequentially output from the preprocessing section 320. Details of the AF control will be described later. The light amount decision section 370 decides a target light amount of the light source based on images sequentially output from the postprocessing section 340, and sequentially outputs the target light amount of the light source to the light source control section 510.

The display section 400 sequentially displays the images output from the postprocessing section 340. That is, the display section 400 displays a video including images with an extended depth of field as frame images. The display section 400 is, for example, a liquid crystal display, an electro-luminescence (EL) display or the like.

The light source device 500 includes a light source control section 510 and a light source 520. The light source control section 510 controls a light amount of the light source 520 in accordance with the target light amount of the light source sequentially output from the light amount decision section 370. The light source 520 emits illumination light. The light source 520 may be a xenon light source, an LED, or a laser light source. Alternatively, the light source 520 may be another light source, and an emission method is not specifically limited.

3. Detailed Description of AF Control

Subsequently, specific examples of the AF control of the present embodiment will be described. First, a description will be given of the first AF control mode using both of the FAR image and the NEAR image, and the second AF control mode using either of the FAR image or the NEAR image. Thereafter, a description will be directed to the switching process between the first AF control mode and the second AF control mode, and a modification of the AF control. Note that a contrast value described below is one example of the AF evaluation value, and may be replaced with another AF evaluation value.

3.1 First AF Control Mode

In a case where the object image is formed at the center position between the image sensor F and the image sensor N, a contrast value of the FAR image and that of the NEAR image are almost equal. Hence, to form the object image at the center position between the image sensor F and the image sensor N, the AF control section 360 is only required to adjust the position of the focus lens while monitoring the contrast value of the FAR image and that of the NEAR image. Also in a case where the target position is set to a position other than the center position between the image sensor F and the image sensor N, the AF control section 360 is only required to associate the image formation position of the object image with a relationship between the contrast value of the FAR image and that of the NEAR image, based on a known PSF shape, a previous experiment, or the like, and to adjust the position of the focus lens 111 while monitoring the relationship between the contrast value of the FAR image and that of the NEAR image.

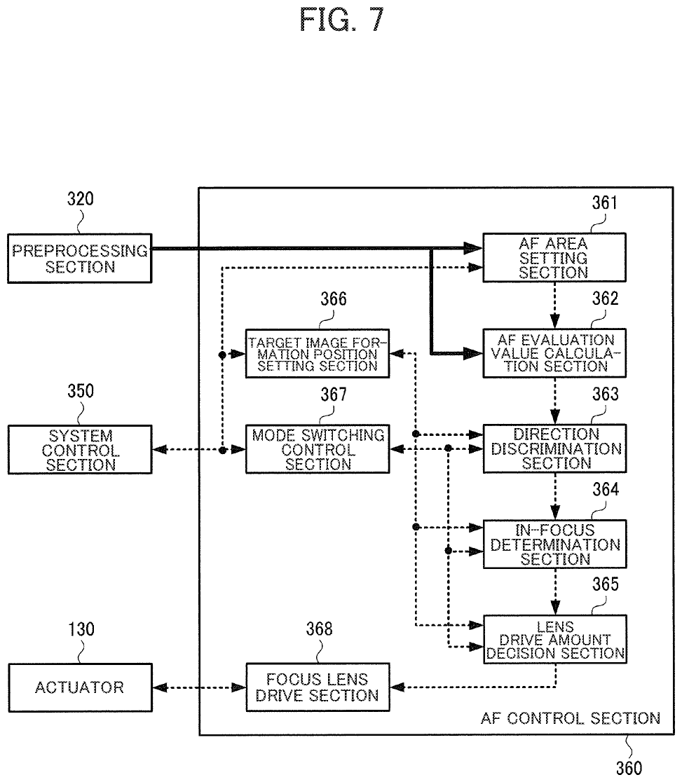

Details of the first AF control mode using both of the contrast value of the FAR image and that of the NEAR image will be described with reference to FIGS. 7 and 8.

FIG. 7 is a configuration diagram of the AF control section 360. The AF control section 360 includes an AF area setting section 361, an AF evaluation value calculation section 362, a direction discrimination section 363, an in-focus determination section 364, a lens drive amount decision section 365, a target image formation position setting section 366, a mode switching control section 367, and a focus lens drive section 368.

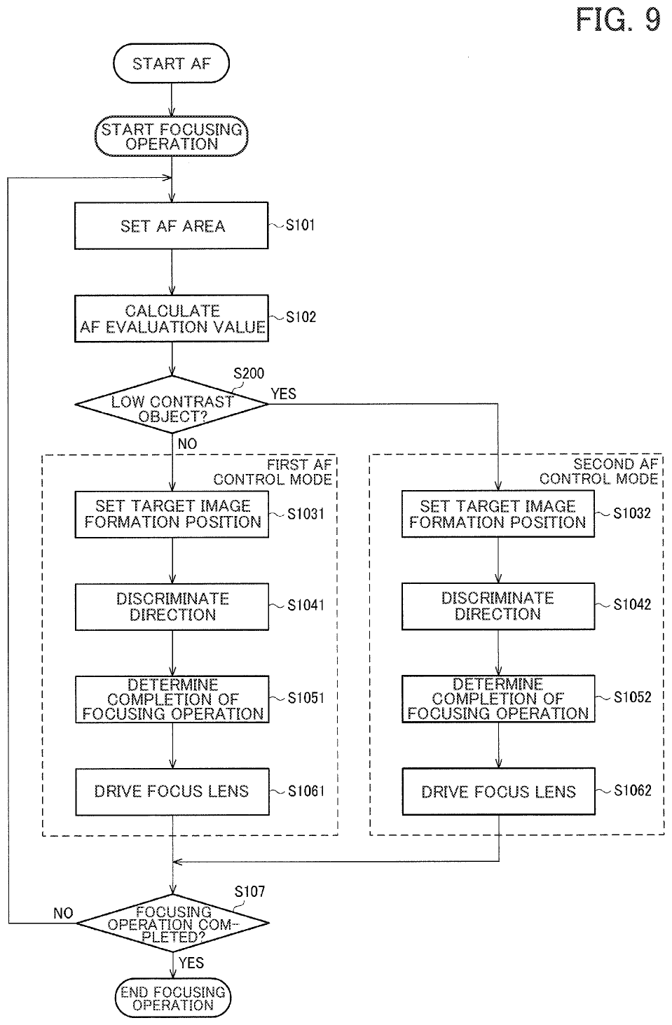

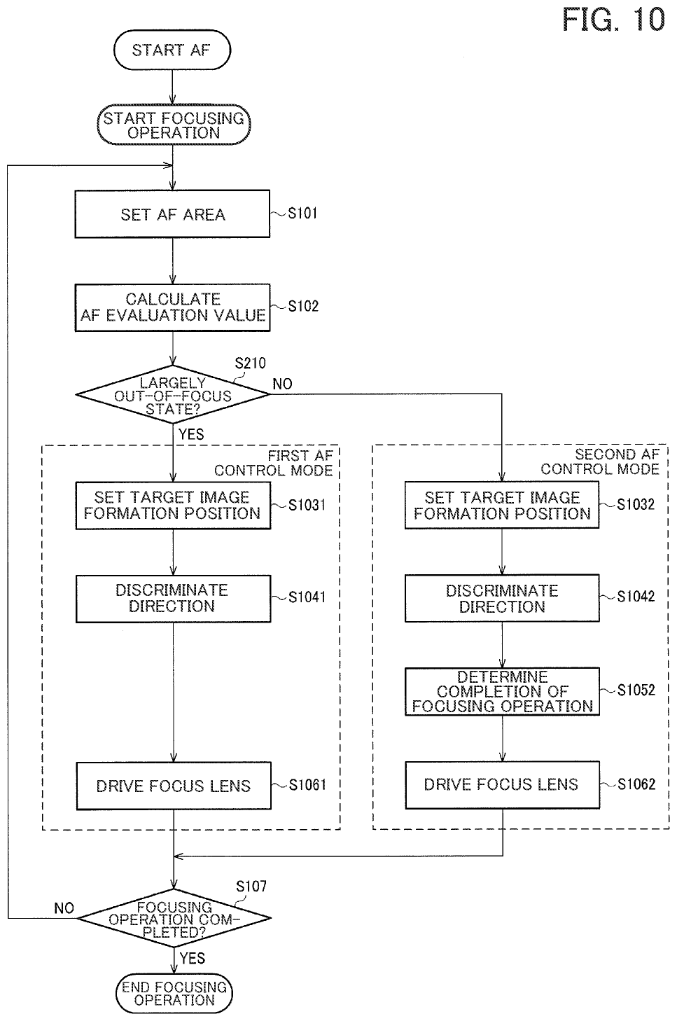

The AF area setting section 361 sets an AF area from which the AF evaluation value is calculated for each of the FAR image and the NEAR image. The AF evaluation value calculation section 362 calculates the AF evaluation value based on a pixel value of the AF area. The direction discrimination section 363 discriminates a drive direction of the focus lens 111. The in-focus determination section 364 determines whether or not a focusing operation has been completed. The lens drive amount decision section 365 decides a drive amount of the focus lens 111. The focus lens drive section 368 drives the focus lens 111 by controlling the actuator 130 based on the decided drive direction and drive amount. The target image formation position setting section 366 sets the target image formation position. The target image formation position is a target position of the image formation position of the target object. The determination made by the in-focus determination section 364 is determination of whether or not the image formation position of the object image has reached the target image formation position. The mode switching control section 367 performs switching of the AF control mode. Note that the AF control mode in the currently described example is the first AF control mode, and details of mode switching will be described later with reference to FIGS. 9 and 10.

FIG. 8 is a flowchart describing the AF control. The AF control starts with the focusing operation. In the focusing operation, the AF area setting section 361 first sets respective AF areas at identical positions with respect to the FAR image and the NEAR image sequentially output from the preprocessing section 320 (S101). For example, the AF area setting section 361 sets each AF area based on information (such as the position or size of the AF area) set by the user from the external I/F section 200. Alternatively, the AF area setting section 361 may detect a lesion using an existing lesion detection function or the like, and may automatically set an area including the detected lesion as the AF area. The AF area is an area in which an image of the target object is captured.

The AF evaluation value calculation section 362 calculates two AF evaluation values corresponding respectively to the FAR image and the NEAR image that are sequentially output from the preprocessing section 320 (S102). The AF evaluation value is a value that increases in accordance with a focusing degree of the object in the AF area. The AF evaluation value calculation section 362 calculates the AF evaluation value by, for example, applying a bandpass filter to each pixel in the AF area and accumulating output values of the bandpass filter. In addition, the calculation of the AF evaluation value is not limited to calculation using the bandpass filter, and a variety of known methods can be employed. The AF evaluation value calculated based on the AF area in the FAR image is hereinafter referred to as an AF evaluation value F, and the AF evaluation value calculated based on the AF area in the NEAR image is hereinafter referred to as an AF evaluation value N.

The target image formation position setting section 366 sets target image formation position information indicating the target image formation position (S103). The target image formation position information is a value indicating a relationship between the AF evaluation value F and the AF evaluation value N. The relationship between the AF evaluation value F and the AF evaluation value N is, for example, ratio information, but may be information indicating another relationship such as difference information. The ratio information or difference information mentioned herein is not limited to information about a simple ratio or difference, and can be extended to various kinds of information based on the ratio or difference. For example, in a case where the target image formation position is set at the center position between the image sensor F and the image sensor N and where the ratio information between the AF evaluation value F and the AF evaluation value N is set as the target image formation position, the target image formation position is one. The target image formation position information may be a freely-selected fixed value, or may be adjusted in accordance with user's preference from the external I/F section 200.

The direction discrimination section 363 discriminates the in-focus direction based on the AF evaluation value F, the AF evaluation value N, and the target image formation position information (S104). The in-focus direction is a drive direction of the focus lens 111 to bring the image formation position of the target object close to the target image formation position. For example, in a case where the target image formation position information is one, the direction discrimination section 363 compares the AF evaluation value F and the AF evaluation value N to determine which is smaller, and discriminates the in-focus direction based on the determination of the smaller value. For example, if the AF evaluation value F is larger than the AF evaluation value N, the in-focus direction will be the drive direction of the focus lens 111 to bring the image formation position close to the image sensor N. In a broad sense, the direction discrimination section 363 calculates, for example, a value indicating current image formation position (image formation position information), and sets, as the in-focus direction, the drive direction of the focus lens 111 to bring the image formation position information close to the target image formation position information. The image formation position information is information similar to the target image formation position information. For example, in a case where the target image formation position information is ratio information between the AF evaluation value F and the AF evaluation value N, the image formation position information is ratio information between the current AF evaluation value F and the current AF evaluation value N.

The in-focus determination section 364 determines whether or not the focusing operation has been completed, based on the target image formation position information and the image formation position information (S105). For example, the in-focus determination section 364 determines completion of the focusing operation when a difference between the target image formation position information and the image formation position information is determined as being equal to or less than a predetermined threshold. Alternatively, the in-focus determination section 364 may determine completion of the focusing operation when a difference between a value 1 and a ratio of the target image formation position information and the image formation position information is equal to or less than the predetermined threshold.

The lens drive amount decision section 365 decides the drive amount of the focus lens 111, and the focus lens drive section 368 drives the focus lens 111 based on a result of the direction discrimination and the drive amount (S106). The drive amount of the focus lens 111 may be a predetermined value, or may be decided based on the difference between the target image formation position information and the image formation position information. Specifically, in a case where the difference between the target image formation position information and the image formation position information is equal to or greater than the predetermined threshold, which means that the current image formation position is considerably distant from the target image formation position, the lens drive amount decision section 365 sets a large drive amount. In a case where the difference between the target image formation position information and the image formation position information is equal to or less than the threshold, which means that the current image formation position is close to the target image formation position, the lens drive amount decision section 365 sets a small drive amount. Alternatively, the lens drive amount decision section 365 may decide the drive amount based on the ratio of the target image formation position information and the image formation position information. Additionally, in a case where completion of the focusing operation has been determined by the in-focus determination section 364 in S105, the drive amount is set to zero. Such control enables setting of an appropriate lens drive amount in accordance with an in-focus state, thereby enabling high-speed AF control.

In a case where completion of the focusing operation has been determined in S105 (when a determination result in S107 is Yes), the AF control section 360 ends the focusing operation and transitions to a wait operation. In a case where the focusing operation has not been completed (when a determination result in S107 is No), the AF control section 360 performs the control from S101 again for each frame.

When the wait operation starts, the AF control section 360 detects a scene change (S201). For example, the AF control section 360 calculates a degree of change over time in AF evaluation value, luminance information about an image, color information, or the like, from either one or both of the two images sequentially output from the preprocessing section 320. When the degree of change over time is equal to or larger than a predetermined degree, the AF control section 360 determines that the scene change has been detected. Alternatively, the AF control section 360 may detect the scene change by calculating a degree of movement of the insertion section 100 or a degree of deformation of a living body serving as the object, by means of movement information about the image, an acceleration sensor (not shown), a distance sensor (not shown), or the like.

In a case where the scene change has been detected (when a determination result in S202 is Yes), the AF control section 360 ends the wait operation and transitions to the focusing operation. In a case where no scene change has been detected (when a determination result in S202 is No), the AF control section 360 performs the control from S201 again for each frame.

As described above, the AF control section 360 of the present embodiment controls the position of the focus lens 111 to be a position at which the first AF evaluation value calculated from the first image and the second AF evaluation value calculated from the second image are determined as having a given relationship. One of the first AF evaluation value and the second evaluation value corresponds to the AF evaluation value N, and the other thereof corresponds to the AF evaluation value F. This control achieves an optimum depth of field range in the combined image with an extended depth of field, based on the relationship between the two AF evaluation values. More specifically, this control can achieve a state in which the image of the target object is formed between the first position corresponding to one of the image sensor N and the image sensor F, and the second position corresponding to the other thereof.

Specifically, the AF control section 360 further includes the direction discrimination section 363 that discriminates the in-focus direction, as illustrated in FIG. 7. In the first AF control mode, the direction discrimination section 363 discriminates the in-focus direction based on the relationship between the first AF evaluation value and the second AF evaluation value. Such control enables discrimination of the direction in a period of time corresponding to one frame, and achieves higher-speed AF control in comparison with known direction discrimination techniques using wobbling or the like.

In addition, the AF control section 360 further includes the lens drive amount decision section 365 that decides the drive amount of the focus lens 111. In the first AF control mode, the lens drive amount decision section 365 decides the drive amount based on the relationship between the first AF evaluation value and the second AF evaluation value. This enables flexible decision of the drive amount in consideration of the relationship between the current image formation position and the target image formation position.

In addition, the AF control section 360 further includes the in-focus determination section 364 that determines whether or not the focusing operation has been completed. In the first AF control mode, the in-focus determination section 364 determines whether or not the focusing operation has been completed based on the relationship between the first AF evaluation value and the second AF evaluation value. The conventional contrast AF or the like requires searching for the peak of the AF evaluation value, and a condition for the conventional in-focus determination is, for example, to detect switching of the in-focus direction a predetermined number of times. The method of the present embodiment, on the other hand, enables the in-focus determination in a period of time corresponding to fewer frames, or one frame in a more limited sense, and thus achieves high-speed AF control.

Note that the AF control section 360 may control the position of the focus lens 111 to be a position determined to form the object image of the target object at the center position between the first position corresponding to the image sensor F and the second position corresponding to the image sensor N. For example, the AF control section 360 controls the position of the focus lens 111 to be such a position that the PSF of the target object is A3 as illustrated in FIG. 2. The center position represents a position at which a distance from the first position and a distance from the second position are substantially equal to each other. With this configuration, the combined depth of field has a range indicated by B3 in FIG. 2, having a width B31 on the far point side and a width B32 on the near point side, with the position of the target object taken as a reference, thereby ensuring a balanced setting. In a case of using the center position, the relationship between the two AF evaluation values is a relationship that establishes a ratio of one, a difference of zero, or a similar relationship.

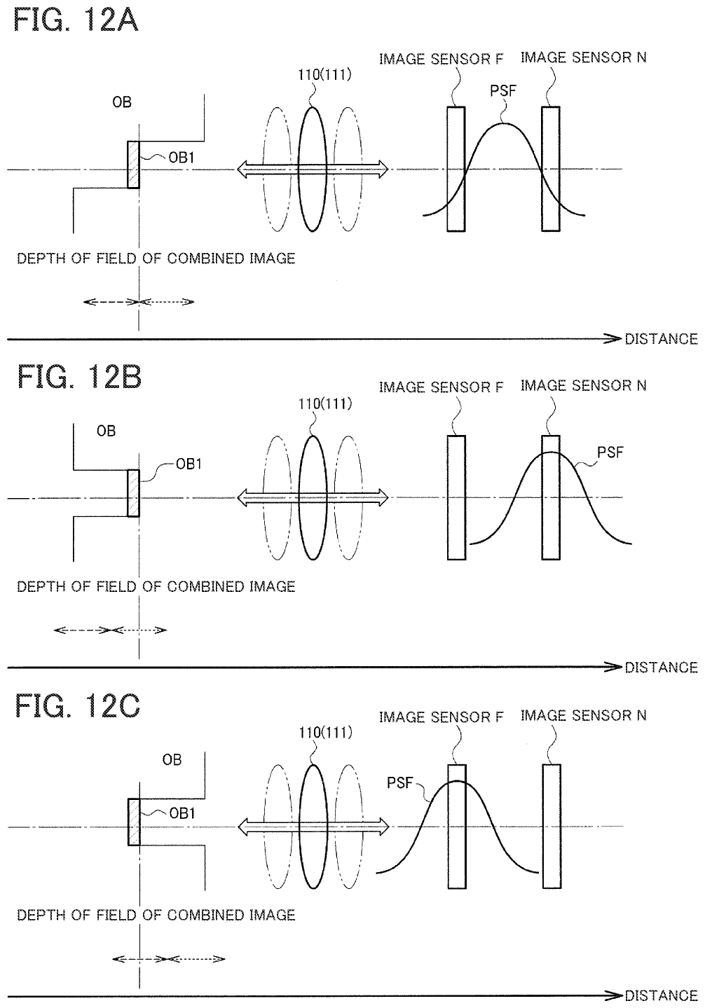

However, the range of a desirable combined depth of field may change with a type of target object, observation situation, user's preference, or the like. Hence, the target image formation position may be another position between the first position and the second position. To be more specific, the AF control section 360 may control the position of the focus lens 111 to be a position determined to form the object image of the target object, at any one of the first position corresponding to the image sensor that acquires the first image, the second position corresponding to the image sensor that acquires the second image, and the position between the first position and the second position. That is, the present embodiment does not prevent the object image of the target object from being formed either at the position corresponding to the image sensor F or at the position corresponding to the image sensor N. This configuration enables flexible setting of the target image formation position. For example, as described later with reference to FIGS. 12B and 12C, when an object shape satisfies a given condition, target image formation position information to be set by the target image formation position setting section 366 indicates a position on the image sensor as the target image formation position.

3.2 Second AF Control Mode

A distance between the image sensor F and the image sensor N is a design value, and thus is a known value. A relationship between a moving amount of the focus lens 111 and a moving amount of the image formation position is also a design value, and thus is a known value. Hence, the AF control section 360 can perform the following control to achieve the optimum depth of field range in the combined image with an extended depth of field. First, the AF control section 360 forms the object image on either of the image sensor F or the image sensor N using the known AF method. As the known AF method, various kinds of methods such as the contrast AF and phase difference AF can be employed. Thereafter, the AF control section 360 controls the position of the focus lens 111 to be a position determined to form the object image at a freely-selected intermediate position between the image sensor F and the image sensor N.

That is, the AF control section 360 includes, as the AF control mode, the second AF control mode of performing the AF control using either of the first AF evaluation value or the second AF evaluation value. By using the second AF control mode, the imaging device 10 that simultaneously captures two images that are different in in-focus object plane position can appropriately set the depth of field range of the combined image while employing the AF control method similar to the conventional method.

Processing in the second AF control mode is similar to that illustrated in FIG. 8, except that the target image formation position to be set by the target image formation position setting section 366 in S103 is an adjusted position of the focus lens 111. For example, the target image formation position setting section 366 sets an amount of adjustment of the focus lens 111 in order to adjust the position of the focus lens 111, after the focus is achieved in either of the image sensor F or the image sensor N. The amount of adjustment involves the drive direction and the drive amount.

Processing in S104 and S105 is similar to that of the known AF control. For example, the AF evaluation value calculation section 362 calculates two AF evaluation values F based on two FAR images acquired in two different timings. Based on a process of comparing the two AF evaluation values, the direction discrimination section 363 discriminates the in-focus direction for bringing the image formation position of the target object onto the image sensor F (S104). In addition, when detection of the peak of the AF evaluation value is determined, the in-focus determination section 364 determines that the focusing operation has been completed (S105). For example, the in-focus determination section 364 determines that the focusing operation has been completed, in a case where switching of the in-focus direction has been detected a predetermined number of times. While the description has been given of the example of forming the object image on the image sensor F using the FAR images, the AF control section 360 may use the NEAR images and form the object image on the image sensor N.

In a case where completion of the focusing operation is not determined in S105, the lens drive amount decision section 365 sets the drive amount to move the image formation position to a position on either of the image sensor N or the image sensor F. The drive amount mentioned herein may be a fixed value, or a dynamically changeable value based on the relationship between two AF evaluation values F (or two AF evaluation values N). In addition, in a case where completion of the focusing operation is determined in S105, the lens drive amount decision section 365 sets the drive amount to move the image formation position from a position on either of the image sensor N or the image sensor F to the target image formation position. The drive amount at this time is the drive amount (adjustment amount) set by the target image formation position setting section 366. The focus lens drive section 368 drives the focus lens 111 in accordance with the set drive amount (S106).

As described above, in the second AF control mode, the AF control section 360 controls the position of the focus lens to be a position determined to form the object image of the target object at the first position corresponding to the image sensor that acquires the first image, and thereafter controls the position of the focus lens 111 to be a position determined to move the image formation position of the object image by a predetermined amount in a direction toward the second position corresponding to the image sensor that acquires the second image. Specifically, in the second AF control mode, the AF control section 360 controls the lens position of the focus lens 111 to the position to form the object image of the target object at the position (P1 in the example illustrated in FIG. 2) corresponding to the image sensor F that acquires the FAR image, and thereafter controls the lens position of the focus lens 111 to the position to move the image formation position of the object image by the predetermined amount in the direction toward the position (P2) corresponding to the image sensor N that acquires the NEAR image. Alternatively, in the second AF control mode, the AF control section 360 controls the lens position of the focus lens 111 to a position to form the object image of the target object at the position (P2) corresponding to the image sensor N that acquires the NEAR image, and thereafter controls the lens position of the focus lens 111 to a position to move the image formation position of the object image by a predetermined amount in a direction toward the position (P1) corresponding to the image sensor F that acquires the FAR image. Such control enables appropriate setting of the depth of field range of the combined image while employing the AF control method similar to the conventional method.

3.3 Process of Switching AF Control Mode

While the description has been given of the processing in the first AF control mode and the processing in the second AF control mode, the AF control mode is not necessarily fixed to either of the first AF control mode or the second AF control mode.

The AF control section 360 may perform switching control between the first AF control mode and the second AF control mode. As illustrated in FIG. 7, the AF control section 360 further includes the mode switching control section 367. The mode switching control section 367 switches between the first AF control mode of performing the AF control using both of the AF evaluation value F and the AF evaluation value N and the second AF control mode of performing the AF control using either of the AF evaluation value F or the AF evaluation value N, in accordance with a feature of the object or an image formation state of the optical system.

In this case, all of the steps corresponding to S103 to S106 may be switched over as described later with reference to FIG. 9, or a part of those steps may be switched over as described later with reference to FIG. 10. Selecting an optimum AF control mode in accordance with the feature of the object and the image formation state of the optical system ensures high-speed, high-accuracy AF control.

For example, in a case where the object is an extremely low contrast object, the difference between the AF evaluation value F and the AF evaluation value N is extremely small, regardless of the image formation state of the optical system, so that high-accurate AF control may be impossible in the first AF control mode. To cope with this case, for example, the mode switching control section 367 first determines whether or not the object is a low contrast object. When the object is determined as the low contrast object, the mode switching control section 367 switches to the second AF control mode. For example, the mode switching control section 367 may determine that the object is the low contrast object in a case where both of the AF evaluation value F and the AF evaluation value N are equal to or less than a threshold. Furthermore, the mode switching control section 367 may make a determination of the low contrast object, based on an additional condition that is determined from a relationship between the AF evaluation value F and the AF evaluation value N. For example, the mode switching control section 367 may determine that the object is the low contrast object, in a case where the difference between the AF evaluation value F and the AF evaluation value N is equal to or less than a threshold, or in a case where a ratio between the AF evaluation value F and the AF evaluation value N is close to one.

FIG. 9 is a flowchart describing the focusing operation in a case where switching control is performed based on the determination of whether or not the object is the low contrast object. S101 and S102 in FIG. 9 are similar to those in FIG. 8. Subsequently, the AF control section 360 determines whether or not the target object is the low contrast object (S200). In a case where the target object is not determined as the low contrast object (when a determination result in S200 is No), the AF control section 360 performs the AF control in the first AF control mode. That is, the target image formation position setting section 366 sets the target image formation position using the relationship between the AF evaluation value F and the AF evaluation value N (S1031). The direction discrimination section 363 determines the in-focus direction, based on the relationship between the AF evaluation value F and the AF evaluation value N (S1041). The in-focus determination section 364 determines whether or not the focusing operation has been completed, based on the relationship between the AF evaluation value F and the AF evaluation value N (S1051). The lens drive amount decision section 365 decides a drive amount of the focus lens 111 based on a result of the direction discrimination and a result of the in-focus determination, and the focus lens drive section 368 drives the focus lens 111 in accordance with the drive amount (S1061).

On the other hand, in a case where the target object is determined as the low contrast object (when a determination result in S200 is Yes), the AF control section 360 performs the AF control in the second AF control mode. That is, the target image formation position setting section 366 sets an adjustment amount of the focus lens 111 after the object image is formed on either of the image sensor F or the image sensor N (S1032). The direction discrimination section 363 discriminates the in-focus direction, using a direction discrimination method in the known contrast AF (S1042). The in-focus determination section 364 determines whether or not the focusing operation has been completed, using an in-focus determination method in the known contrast AF (S1052). The lens drive amount decision section 365 decides a drive amount of the focus lens, and the focus lens drive section 368 drives the focus lens 111 based on a result of the direction discrimination and the drive amount (S1062). In the second AF control mode, in a case where the in-focus determination section 364 determines completion of the focusing operation in S1052, the focus lens drive section 368 drives the focus lens 111 based on the adjustment amount of the focus lens 111 that is set in S1032, regardless of the result of the direction discrimination.

The AF control illustrated in FIG. 9 ensures high-accuracy AF control also for the low contrast object.

Additionally, when the optical system is largely out of focus, an operation in the second AF control mode using wobbling drive or the like may not be able to discriminate the direction accurately. In this context, “largely out of focus” represents a state in which a focusing degree of the object in the captured image is significantly low. In this case, the mode switching control section 367 first determines whether or not the optical system is a largely out-of-focus state. In a case where the largely out-of-focus state is determined, the mode switching control section 367 performs switching control to the first control mode. For example, the mode switching control section 367 determines that the optical system is in the largely out-of-focus state in a case where both of the following conditions are met: both of the AF evaluation value F and the AF evaluation value N are equal to or less than a threshold; and a difference between the AF evaluation value F and the AF evaluation value N is equal to or greater than a threshold. Alternatively, the mode switching control section 367 determines that the optical system is in the largely out-of-focus state in a case where the ratio of the AF evaluation value F and the AF evaluation value N is high.

FIG. 10 is a flowchart describing the focusing operation in a case where switching control is performed based on the determination of whether or not the optical system is in the largely out-of-focus state. S101 and S102 in FIG. 10 are similar to those in FIG. 8. Subsequently, the AF control section 360 determines whether or not the optical system is the largely out-of-focus state (S210). In a case where the optical system is determined to be in the largely out-of-focus state (when a determination result in S210 is Yes), the AF control section 360 performs the AF control in the first AF control mode. In a case where the optical system is not determined to be in the largely out-of-focus state (when a determination result in S210 is No), the AF control section 360 performs the AF control in the second AF control mode.

Note that the AF control section 360, when operating in the first AF control mode, does not perform the in-focus determination corresponding to S1051 in FIG. 9. Since the largely out-of-focus state is eliminated as the optical system approaches the in-focus state, the in-focus determination in the first AF control mode offers little advantage. The control in the other steps is just as described above. According to such AF control, the AF control section 360 performs the AF control in the first AF control mode that enables high-speed direction discrimination in a case where the optical system is largely out of focus, and thereafter, as the optical system approaches the in-focus state, the AF control section 360 performs the AF control in the second AF control mode that enables the high-accuracy focusing operation. Such AF control can achieve high-speed, high-accuracy AF control.

As described above, the AF control section 360 performs a low contrast object determination process of determining whether or not the target object is the low contrast object that has a lower contrast than a given reference, based on the first AF evaluation value and the second AF evaluation value. Then, based on the low contrast object determination process, the AF control section 360 performs a switching process between the first AF control mode and the second AF control mode. Alternatively, the AF control section 360 performs a largely out-of-focus determination process of determining whether or not the optical system is in the largely out-of-focus state in which the focusing degree of the target object is lower than a given reference, based on the first AF evaluation value and the second AF evaluation value. Then, based on the largely out-of-focus determination process, the AF control section 360 performs a switching process between the first AF control mode and the second AF control mode. Note that the reference for determination of the low contrast object and the reference for determination of the largely out-of-focus state may be fixed values or dynamically changeable values.

This configuration enables selection of an appropriate AF control mode in accordance with the feature of the target object or an imaging state. At this time, using both of the first AF evaluation value and the second AF evaluation value enables higher-speed determination for execution of switching control in comparison with the conventional method that involves wobbling control or the like. However, in the low contrast object determination process and the largely out-of-focus determination process, the present embodiment does not exclude a modification using either of the first AF evaluation value or the second AF evaluation value. Further, in the example described herein, the switching between the first AF control mode and the second AF control mode relies on the determination of whether or not the object is the low contrast object or the determination of whether or not the optical system is in the largely out-of-focus state. However, the determination for the switching is not limited thereto, and the switching may rely on another determination.

3.4 Modification of AF Control

In addition, the AF control in the first AF control mode is not limited to the above-mentioned method of repeating the direction discrimination and the in-focus determination. For example, in the first AF control mode, the AF control section 360 controls the position of the focus lens 111 to be between a position of the focus lens 111 corresponding to the peak of the first AF evaluation value and a position of the focus lens 111 corresponding to the peak of the second evaluation value. Note that the peak of the AF evaluation value is a maximum of the AF evaluation value.

Specifically, the AF control section 360 first acquires the FAR and NEAR images while driving (scanning) the focus lens 111 in a given range. Based on the FAR image and the NEAR image captured at respective focus lens positions, the AF control section 360 calculates contrast values, and thereby obtains a relationship between the focus lens position and the contrast value of the FAR image as well as a relationship between the focus lens position and the contrast value of the NEAR image. The AF control section 360 detects the focus lens position at the peak of each contrast value. Thereafter, the AF control section 360 adjusts the focus lens position at a freely-selected position between the focus lens positions corresponding to the two peaks.

The focus lens position at the peak of the contrast value of the FAR image is a focus lens position at which an image of the target object is formed on the image sensor F. The focus lens position at the peak of the contrast value of the NEAR image is a focus lens position at which an image of the target object is formed on the image sensor N. With this configuration, the technique of scanning the focus lens 111 in the given range enables the image formation position of the target object to be set between the first position and the second position. Consequently, the optimum depth of field range can be achieved in the combined image with an extended depth of field.

Alternatively, the AF control section 360 may perform the AF control in the second AF control mode, based on peak detection by scan-driving.

3.5 Estimation of Object Shape

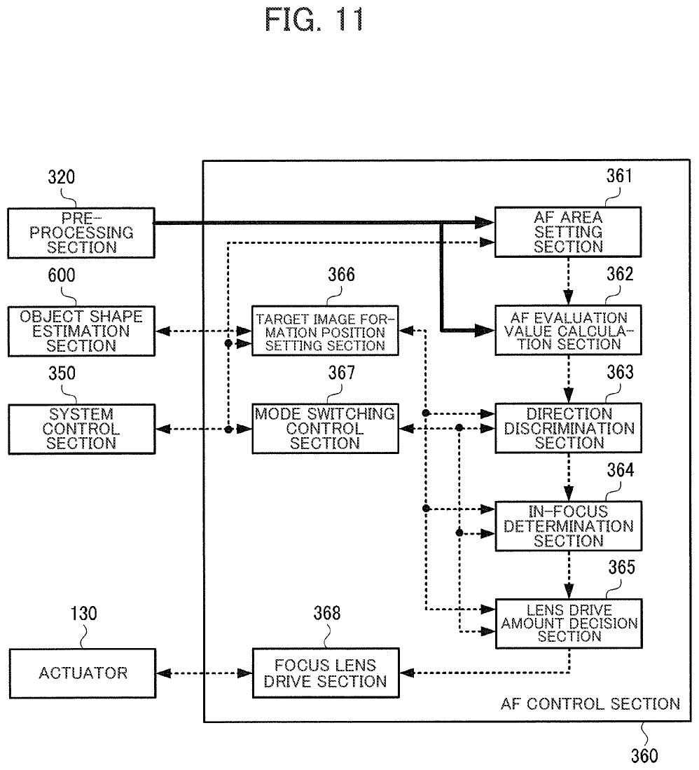

FIG. 11 illustrates another configuration example of the endoscope apparatus 12, which is one example of the imaging device 10 in accordance with the present embodiment. The endoscope apparatus 12 further includes an object shape estimation section 600 that estimates the shape of the target object and the shape of other objects surrounding the target object. In addition, the endoscope apparatus 12 includes the target image formation position setting section 366, which sets the target image formation position based on the object shape estimated in the object shape estimation section 600. The AF control section 360 then controls the position of the focus lens 111 to be a position determined to form the object image of the target object at the target image formation position set in the target image formation position setting section 366. Such control enables acquisition of the combined image in which an optimum range is in focus in accordance with the shape of the object.

FIGS. 12A to 12C are diagrams exemplifying relationships between the object shapes and the desirable depth of field ranges. The example described with reference to FIG. 2, etc. assumes that the object is present in each of the direction near the objective optical system and the direction far from the objective optical system with respect to the target object, as illustrated FIG. 12A. In this case, to acquire the combined image in which the object is in focus in a balanced manner, the target image formation position is set at the position between the image sensor F and the image sensor N.

However, in a probable scene during an actual endoscopic examination, the object is present only in a direction far from the target object with respect to the objective optical system 110. A conceivable example of such a scene is to observe a polyploid lesion in a direction close to the front side, as illustrated in FIG. 12B. In a case where the object shape estimation section 600 estimates that the object has such a shape, the target image formation position setting section 366 sets the target image formation position at a position nearer the position corresponding to the image sensor N. More specifically, the target image formation position setting section 366 sets target image formation position information that indicates the position of the image sensor N as the target image formation position. This configuration enables acquisition of the combined image in which the polyploid object is in focus in a wide range.

In another probable scene during the endoscopic examination, the object is present only in a direction near the target object with respect to the objective optical system 110. A conceivable example of such a scene is to observe a depressed lesion in a direction close to the front side, as illustrated in FIG. 12C. In a case where the object shape estimation section 600 estimates that the object has such a shape, the target image formation position setting section 366 sets the target image formation position at a position nearer the position corresponding to the image sensor F. More specifically, the target image formation position setting section 366 sets the target image formation position information that indicates the image sensor F as the target image formation position. This configuration enables acquisition of the combined image in which the depressed object is in focus in a wide range.

Further, in a case of setting the target image formation position information at the position between the image sensor F and the image sensor N, the target image formation position setting section 366 may also set the target image formation position information adaptively based on the object shape estimated in the object shape estimation section 600.

The object shape estimation section 600 estimates the object shape, for example, by utilizing information such as luminance distribution and color distribution from an image output from the preprocessing section 320. Alternatively, the object shape estimation section 600 may estimate the object shape using known shape estimation techniques such as Structure from Motion (SfM) and Depth from Defocus (DfD). Alternatively, the endoscope apparatus 12 may further include a device (not shown) that is capable of known distance measurement or shape measurement, such as a twin-lens stereo photography device, a light field photography device, or a distance measuring device by pattern projection or Time of Flight (ToF), and the object shape estimation section 600 may estimate the object shape based on an output from such a device. As described above, the processing in the object shape estimation section 600 and the configuration to implement the processing can be modified in various manners.

In addition, the method of the present embodiment can be applied to an operation method of the imaging device 10 including the objective optical system 110, the optical path splitter 121, and the image sensor 122. The operation method of the imaging device includes a combining process of selecting an image with a relatively high contrast in predetermined corresponding areas between the first image and the second image to generate a single combined image, and AF control of controlling the position of the focus lens 111 to be a position determined to bring the target object into focus based on at least one of the first image or the second image before the combining process. In addition, the operation method of the imaging device 10 includes the combining process described above and AF control of operating in accordance with a given AF control mode including the first AF control mode to control the position of the focus lens 111 to be a position determined to bring the target object into focus.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An imaging device comprising:
an objective optical system that includes a focus lens for adjusting an in-focus object plane position and acquires an object image;
an optical path splitter that splits the object image into a first optical image and a second optical image different from each other in the in-focus object plane position;
an image sensor that captures the first optical image to acquire a first image and the second optical image to acquire a second image; and
a processor including hardware,
the processor performing
a combining process of selecting an image with a relatively high contrast in predetermined corresponding areas between the first image and the second image to generate a single combined image, and
an Auto Focus (AF) control of controlling a position of the focus lens to be a position determined to bring a target object into focus,
the processor controlling the position of the focus lens to be a position determined to form the object image of the target object at a position between a first position corresponding to the image sensor for acquiring the first image and a second position corresponding to the image sensor for acquiring the second image, based on the first image and the second image before the combining process.

2. The imaging device as defined in claim 1,
wherein the processor estimates an object shape of the target object and an object shape of a peripheral object around the target object, sets a target image formation position based on the estimated object shapes, and controls the position of the focus lens to a position determined to form the object image of the target object at the set target image formation position.

3. The imaging device as defined in claim 1,
wherein the processor controls the position of the focus lens to a position determined to form the object image of the target object at a center position between the first position and the second position.

4. The imaging device as defined in claim 1,
wherein the processor controls the position of the focus lens to be a position determined to form the object image of the target object at the first position, and thereafter controls the position of the focus lens to a position determined to move an image formation position of the object image by a predetermined amount in a direction toward the second position.

5. The imaging device as defined in claim 1,
wherein the processor controls the position of the focus lens to a position determined to bring a first AF evaluation value calculated from the first image and a second AF evaluation value calculated from the second image into a given relationship.

6. The imaging device as defined in claim 1,
wherein the processor controls the position of the focus lens to a position between a position of the focus lens corresponding to a peak of a first AF evaluation value calculated from the first image and a position of the focus lens corresponding to a peak of a second AF evaluation value calculated from the second image.

7. An endoscope apparatus comprising:
an objective optical system that includes a focus lens for adjusting an in-focus object plane position and acquires an object image;
an optical path splitter that splits the object image into a first optical image and a second optical image different from each other in the in-focus object plane position;
an image sensor that captures the first optical image to acquire a first image and the second optical image to acquire a second image; and
a processor including hardware,
the processor performing
a combining process of selecting an image with a relatively high contrast in predetermined corresponding areas between the first image and the second image to generate a single combined image, and
an Auto Focus (AF) control of controlling a position of the focus lens to be a position determined to bring a target object into focus,
the processor controlling the position of the focus lens to be a position determined to form the object image of the target object at a position between a first position corresponding to the image sensor for acquiring the first image and a second position corresponding to the image sensor for acquiring the second image, based on the first image and the second image before the combining process.

8. An operation method of an imaging device, the imaging device including:
an objective optical system that includes a focus lens for adjusting an in-focus object plane position and acquires an object image;
an optical path splitter that splits the object image into a first optical image and a second optical image different from each other in the in-focus object plane position; and
an image sensor that captures the first optical image to acquire a first image and the second optical image to acquire a second image,
the method comprising:
a combining process of selecting an image with a relatively high contrast in predetermined corresponding areas between the first image and the second image to generate a single combined image; and
an Auto Focus (AF) control of controlling a position of the focus lens to be a position determined to form the object image of a target object at a position between a first position corresponding to the image sensor for acquiring the first image and a second position corresponding to the image sensor for acquiring the second image, based on the first image and the second image before the combining process.
